(19) United States
(12) Patent Application Publication
(10) Pub. No.: US 2018/0256483 A1
(43) Pub. Date: Sep. 13, 2018

(54) Skincare product containing hermetia illucens extract

(71) Applicant: Chanarong Sangduan, San Sai (TH)

(72) Inventor: Chanarong Sangduan, San Sai (TH)

(21) Appl. No.: 15/981,689

(22) Filed: May 16, 2018

Related U.S. Application Data
(63) Continuation of application No. PCT/TH2015/000080, filed on Nov. 16, 2015.

(51) Int. Cl. A61K 8/92; A61K 8/67; A61K 8/02; A61Q 19/00; C11B 1/10; C11B 1/08

(52) U.S. Cl. CPC A61K 8/927; A61K 8/678; C11B 1/08; A61Q 19/00; C11B 1/10; A61K 8/022

(57) ABSTRACT

The present disclosure relates to processes for preparing a skincare product, the process comprising drying a black soldier fly to produce a dried black solder fly; extracting the dried black soldier to produce an oil extract or a fat extract of the black soldier fly; separating the oil extract or the fat extract of the black soldier fly from the mixture to produce a separated oil extract or fat extract of the black soldier fly; sterilizing the separated oil extract or fat extract of the black soldier fly to produce a sterilized oil extract or fat extract of the black soldier fly; and mixing the sterilized oil extract or fat extract of the black soldier fly with additional ingredients to form the skincare product, wherein the pharmaceutical product contains the sterilized oil extract or fat extract of the black solder fly.

### CROSS-REFERENCE TO RELATED APPLICATIONS

This application is a continuation of PCT Application No. PCT/TH2015/000080, filed Nov. 16, 2015. The contents of this application are hereby incorporated by reference in its entirety.

### FIELD OF THE DISCLOSURE

The present disclosure relates to a skincare product containing a black soldier fly (hermetia illucens) extract and a preparation process thereof.

### BACKGROUND OF THE DISCLOSURE

The black soldier fly, Hermetia illucens, is one of the most important species proposed as a converter of organic waste due to rapid food intake of up to 500 mg of fresh matter per day. Since the black soldier fly naturally feeds on organic wastes, converting biomass nutrients into its own biomass, it is a promising source of nutrients like proteins, lipids, and minerals. For example, the black soldier fly is used as feed for a number of species, including fish, chicken, and pigs, as a pet food, and as an alternative feedstock for the production of biodiesel. The black soldier fly is also a source of chitin and its derivatives, having great commercial value due to its numerous applications: food, cosmetics, pharmaceuticals, textiles, etc. Therefore, the development of applications using biomolecules from the black soldier fly is an issue of growing importance, to obtain high value marketable products and provide further economic benefits.

CN 102585035 A disclosed a method for preparing chitosan extracted from fly black water comprising steps of using hydrochloric acid for decalcification, dilute alkalis for protein removal, sodium hypochlorite for depigmentation, and removing acetyl compound to obtain chitosan. The chitosan prepared by the method can be applied to environmental protection, medical treatment, foods, textiles, and agriculture.

CN 101880593 A and CN 106701313 A disclosed the extraction method of the black soldier fly using a hot press method and ultrasound-assisted soxhlet extraction respectively, in order to obtain an oil for use as a nutrient supplement, and chemical and biodiesel applications.

CN 105456155 A disclosed a method for extracting oil from hermetia illucens larvae by supercritical CO₂ extraction. The method has the advantage of high extracting efficiency, is free of pollution and residues, and is environmentally friendly. The extracted oil is rich in polyunsaturated fatty acid, which can be used as a raw cosmetics material.

However, there is not any prior art disclosing use of black soldier fly extract as an active ingredient in a skincare product. Therefore, this disclosure relates, in some embodiments, to a skincare product containing black soldier fly (hermetia illucens) extract with good properties. The present disclosure relates, in some embodiments, to a process for preparing a skincare product containing said extract.

### SUMMARY

According to some embodiments, the present disclosure relates to a skincare product containing black soldier fly (hermetia illucens) extract comprising:

oil extract or fat extract of black soldier fly (hermetia illucens) in a range of 1-30% by weight of the skincare product;

vitamin E in a range of 1-5% by weight of the skincare product; and

cosmetic acceptable ingredients q.s or from about 1% to 100% by weight of the skincare product.

According to some embodiments, the present disclosure relates also provides a process for preparing a skincare product comprising:
i) drying the black soldier fly (hermetia illucens) at a temperature of 60-90° C. for 5 minutes to 3 hours;
ii) extracting the dried black soldier fly by solvent extraction, screw press, or supercritical CO₂ extraction;
iii) separating the oil extract or fat extract of black soldier fly (hermetia illucens) from the mixture obtained from step ii) by filtration or centrifugation;
iv) sterilizing the oil extract or fat extract of black soldier fly (hermetia illucens) obtained from step iii) by heating at a temperature less than 90° C.; and
v) mixing the oil extract or fat extract of black soldier fly (hermetia illucens) obtained from step iv) in a range of 1-30% by weight of the skincare product with vitamin E in a range of 1-5% by weight of the skincare product and cosmetically acceptable ingredients q.s to 100% by weight of the skincare product.

### DETAILED DESCRIPTION

The present disclosure relates, according to some embodiments, to a skincare product containing black soldier fly (hermetia illucens) extract, including a process for preparing the skincare product containing said extract. The obtained skincare composition has beneficial properties that improve skin conditions, including revitalizing, smoothing, moisturizing and tightening the skin, which can be explained according to the following specification.

Any aspect showed here aims to include every application of other aspects of this disclosure unless stated otherwise.

Technical terms or scientific terms used herein have definitions as understood by those having an ordinary skill in the art, unless stated otherwise.

Equipment, apparatus, methods, or chemicals mentioned here refer to equipment, apparatus, processes, or chemicals commonly operated or used by those skilled in the art, unless explicitly stated otherwise that they are specifically used in embodiments according to this disclosure.

The use of the singular or plural nouns with the term “comprising” in the claims or in the specification refers to “one” and also “one or more,” “at least one,” and “one or more than one.”

All compositions and/or processes disclosed and claimed are intended to include aspects of the disclosure from actions, operation, modifications, or changes to any parameters without performing significantly different experiments from this disclosure, and obtaining similar objects with same utilities and results of the present disclosure according to persons skilled in the art, although without mentions in claims specifically. Therefore, substitution or similar objects to the present disclosure including minor modifications or changes, which are clearly apparent to persons having ordinary skill in the art should consider the scope, spirit, and concept of the disclosure as appended claims.

Throughout this application, the term “about” is used to indicate that any value presented herein may potentially vary or deviate. Such variation or deviation may result from errors of apparatus, methods used in calculation, or from individual operator implementing apparatus or methods. These include variations or deviations caused by changes of physical properties.

The following shows a detailed description of some embodiments according to the present disclosure without any intention to limit the scope of the disclosure.

According to some embodiments, the present disclosure relates to a skincare product containing black soldier fly (hermetia illucens) extract comprising:

oil extract or fat extract of black soldier fly (hermetia illucens) in a range of 1-30% by weight of the skincare product;

vitamin E in a range of 1-5% by weight of the skincare product; and

cosmetically-acceptable ingredients q.s to 100% by weight of the skincare product.

In some embodiments, the skincare product comprising a black soldier fly (hermetia illucens) extract in a range of 10-30% by weight of the skincare product.

In some embodiments, the oil extract or fat extract of black soldier fly (hermetia illucens) is oil extract or fat extract of black soldier fly larvae (hermetia illucens larvae).

In some embodiments, the oil extract or fat extract of black soldier fly (hermetia illucens) extract comprises 5-10% by weigh of monoglyceride, 1-5% by weigh of diglyceride, 1-5% by weigh of triglyceride, and 80-93% phospholipid.

In some embodiments, vitamin E is in the range of 2-5% by weight of the skincare product.

According to some embodiments, the cosmetically-acceptable ingredients can be active or inactive ingredients selected from but not limited to antioxidants, anti-UV agents, diluents, lubricants, emulsifying agents, thickening agents, preservatives, stabilizers, emollients, humectants, cosmetic makeup ingredients, pH adjusting agents, chelating agents, binders, antimicrobial agents, fragrances or a combination thereof. These ingredients can be adjust so that the present skin product be formulated as a cream or liquid or ointment, or gel.

In some embodiments, the cosmetically-acceptable can be selected from but not limited to water, ethylene glycol, propylene glycol, butylene glycol, dipotassium glycyrrhizate, polyethylene glycol and its derivatives, acrylates copolymers, C₁₀₋₃₀ alkyl acrylate crosspolymers, alkyl esters of cetyl alcohol, squalene, sorbitan monoesters, polyethoxylated sorbitan esters, long chain fatty alcohols, silicone based polymer, propanediol, ethylhexylglycerin, caprylyl glycol, caprylhydroxamic acid, ethylenediaminetetraacetic acid, or a combination thereof.

In some embodiments, the present disclosure relates to a process for preparing a skincare product comprising steps of:
i) drying the black soldier fly at a temperature of 60-90° C. for 5 minutes to 3 hours to produce a dried black solder fly (hermetia illucens);
ii) extracting the dried black soldier fly (hermetia illucens) by solvent extraction, screw press, or supercritical CO₂ extraction to produce an oil extract or a fat extract of the black soldier fly (hermetia illucens);
iii) separating oil extract or fat extract of black soldier fly (hermetia illucens) from the mixture obtained from step ii) by filtration or centrifugation to produce a separated oil extract or fat extract of the black soldier fly (hermetia illucens);
iv) sterilizing the oil extract or fat extract of black soldier fly (hermetia illucens) obtained from step iii) by heating at a temperature less than 90° C. to produce a sterilized oil extract or fat extract of the black soldier fly (hermetia illucens); and
v) mixing the sterilized oil extract or fat extract of the black soldier fly (hermetia illucens) with additional ingredients to form the skincare product, the additional ingredients comprising a vitamin E in a range of 1-5% by weight of the skincare product and a cosmetically-acceptable ingredient in a range of 1-100% by weight of the skincare product, wherein the pharmaceutical product contains the sterilized oil extract or fat extract of the black solder fly (hermetia illucens) is in a range from 1-30% by weight.

In some embodiments, the black soldier fly (hermetia illucens) is black soldier fly (hermetia illucens) larvae.

In some embodiments, in step i), the black soldier fly (hermetia illucens) is dried by heating at a temperature of 60-90° C. for 2-5 hours, or by microwave drying at a temperature of 60-90° C. for 30-60 minutes.

In some embodiments, in step ii), the dried black soldier fly (hermetia illucens) is extracted by a screw press or by a supercritical CO₂ extraction at a pressure of 200-250 bar, and at a temperature of 40-60° C. for 2-3 hours.

In some embodiments, in step iii), the oil extract or fat extract of black soldier fly (hermetia illucens) is separated from the extracted mixture by filtration with a filter having a size of 20-30 μm or

In some embodiments, in step iii), the oil extract or fat extract of black soldier fly (hermetia illucens) is separated from the extracted mixture by centrifugation at 2500-4000 rpm, at a temperature of 25-40° C., for 10-30 minutes.

In some embodiments, in step iv), the obtained oil extract or fact extract is sterilized by heating at a temperature of 60-90° C.

In some embodiments, the process for preparing a skincare product further comprises a step of freezing black soldier fly at a temperature from −5 to −20° C. prior to subjecting to step i).

In some embodiments, the process for preparing a skincare product further comprises a step of grinding or milling the dried black soldier fly obtained from step i) to a powder, prior to subjecting said powder to step ii).

Preferably, the black soldier fly powder has a size less than 1 mm.

Some specific example embodiments of the disclosure may be illustrated by one or more of the examples provided herein without any purpose to limit any scope of the disclosure.

Extraction of a Black Soldier Fly (Hermetia Illucens)

Black soldier flies were washed by water, followed by freezing at a temperature of −5 to −20° C. for about 2 hours. The water used for washing may be room temperature water or cold water. Then, the flies were dried using a hot air oven at a temperature of about 90° C. for about 3 hours. The dried black soldier flies were then extracted using a screw press to obtain an oil extract or fat extract. The obtained oil extract or fat extract was filtered by a filter having a pore size of 20-30 μm, followed by sterilizing at a temperature of about 90° C. in order to obtain the final oil extract or fat extract.

The composition of the oil extract or fat extract of the black soldier fly was analyzed by using a thin layer chromatography-flame ionization detection (TLC-FID). It was found that the oil extract or fat extract from the black soldier fly comprises a monoglyceride at 5-10% by weight of the oil extract or fat extract, a diglyceride at 1-5% by weight of the oil extract or fat extract, a triglyceride at 1-5% by weight of the oil extract or fat extract, and a phospholipid at 80-93% by weight of the oil extract or fat extract. The oil extract or fat extract of the black soldier fly may also further comprise other compounds at 0.5-1% by weight of the oil extract or fat extract. For example, other compounds may comprise fatty acids and cholesterols.

Minimum Inhibitory Concentration (MIC) Test and Minimum Bactericidal Concentration (MBC) Test

The oil extract or fat extract of black soldier fly (hermetia illucens) obtained from black soldier fly larvae was tested for minimum inhibitory concentration (MIC) and minimum bactericidal concentration (MBC) against Candida albicans, Escherichia Coli, Pseudomonas aeruginosa, Stapphylococcus aureus, Kytococcus sendentarius, and Propionibacterium acnes. The results of MIC and MBC tests are shown in Table 1. It was found that the lowest concentration preventing a growth of Candida albicans, Escherichia Coli, Pseudomonas aeruginosa, Stapphylococcus aureus, Kytococcus sendentarius, and Propionibacterium acnes is 50, 25, 50, 25, 25, 12.5% by weight respectively, and the lowest concentration of the black soldier fly extract required to kill Candida albicans, Escherichia Coli, Pseudomonas aeruginosa, Stapphylococcus aureus, Kytococcus sendentarius, and Propionibacterium acnes is 50% by weight.

TABLE 1
Minimum inhibitory concentration (MIC) and minimum bactericidal
concentration (MBC) against Candida albicans, Escherichia Coli,
Psudomonas aeruginosa, Stapphylococcus aureus,
Kytococcus sendentarius, and Propionibacterium acnes.

| | MIC (% w/v) | MBC (% w/v) |
|---|---|---|
| C. albicans, | 50 | 50 |
| E. Coli | 25 | 50 |
| P. aeruginosa | 50 | 50 |
| S. aureus | 25 | 50 |
| K. sendentarius | 25 | 50 |
| P. acnes | 12.5 | 50 |

Antioxidant Activity Test

The oil extract or fat extract of black soldier fly (hermetia illucens) obtained from black soldier fly larvae was tested for antioxidant activity using DPPH assay. The samples of black soldier fly extract were reacted with a solution of DPPH (2,2-diphenyl-1-picryl-hydrazyl-hydrate) in ethanol followed by adding a butylated hydroxytoluene standard solution into the mixture. The changes in color of the samples were read (Absorbance, Abs) at 520 nm using a UV-VIS spectrophotometer. It was found that the antioxidant activity of the black soldier fly extract expressed as SC₅₀ value, a concentration giving 50% scavenging of DPPH radicals of the black soldier fly is 14.43±0.14.

Tyrosinase Inhibition Activity

The oil extract or fat extract of black soldier fly (hermetia illucens) obtained from black soldier fly larvae was tested for tyrosinase inhibition activity using L-DOPA as a substrate. The samples of the black soldier fly extract were added to 50 μL, of L-DOPA (1 mg/ml) followed by 50 μL, of tyrosinase (200 U/ml). The mixtures were monitored to have an absorbance of 492 nm using a UV-VIS spectrophotometer for dopachrome formation in the reaction mixture. Kojic acid was used as a positive control. The IC₅₀ value, a concentration giving 50% inhibition of tyrosinase activity, of the black soldier fly extract is 0.13±0.04.

Moreover, from the toxicity testing, the black soldier fly extract obtained from black soldier fly larvae has an LD50 value of more than 500 mg/kg. From all testing results, the oil extract or fat extract of black soldier fly (hermetia illucens) can be used for preparing a skincare product according to the present disclosure. The product obtained from the present disclosure prepared composition has good stability, good appearance and good properties for penetrating human skin through absorption.

### BEST MODE OF THE DISCLOSURE

Best mode of the disclosure is as disclosed in the detailed description.

What is claimed is:

1. A skincare product comprising an oil extract or a fat extract of a black soldier fly (hermetia illucens) extract, the skincare product comprising:
the oil extract or the fat extract of the black soldier fly (hermetia illucens) in a range of 1-30% by weight of the skincare product;
a vitamin E in a range of 1-5% by weight of the skincare product; and
a cosmetically-acceptable ingredient of 1-100% by weight of the skincare product.

2. The skincare product according to claim 1, wherein the oil extract or the fat extract of the black soldier fly (hermetia illucens) is in a range of 10-30% by weight of the skincare product.

3. The skincare product according to claim 1, wherein the oil extract or the fat extract of the black soldier fly (hermetia illucens) comprises an oil extract or a fat extract of a black soldier fly larvae.

4. The skincare product according to claim 1, wherein the oil extract or the fat extract of the black soldier fly (hermetia illucens) comprises a monoglyceride at 5-10% by weight of the oil extract or the fat extract, a diglyceride at 1-5% by weight of the oil extract or the fat extract, a triglyceride at 1-5% by weight of oil extract or the fat extract, and a phospholipid at 80-93% by weight of the oil extract or the fat extract.

5. The skincare product according to claim 1, wherein the vitamin E is in the range of 2-5% by weight of the skincare product.

6. The skincare product according to claim 1, wherein the cosmetic acceptable ingredient is selected from the group consisting of antioxidants, anti-UV agents, diluents, lubricants, emulsifying agents, thickening agents, preservatives, stabilizers, emollients, humectants, cosmetic makeup ingredients, pH adjusting agents, chelating agents, binders, antimicrobial agents, fragrances, and combination thereof.

7. A process for preparing a skincare product, the process comprising steps of:
(a) drying a black soldier fly (hermetia illucens) at a temperature of 60-90° C. for a time of 5 minutes to 3 hours to produce a dried black solder fly (hermetia illucens);
(b) extracting the dried black soldier fly (hermetia illucens) by a solvent extraction, a screw press, or a supercritical CO₂ extraction to produce an oil extract or a fat extract of the black soldier fly (hermetia illucens) to produce an extracted mixture;
(c) separating the oil extract or the fat extract of the black soldier fly (hermetia illucens) from the extracted mixture by filtration or centrifugation to produce a separated oil extract or fat extract of the black soldier fly (hermetia illucens);
(d) sterilizing the separated oil extract or fat extract of the black soldier fly (hermetia illucens) obtained from step iii) by heating at a sterilizing temperature of less than 90° C. to produce a sterilized oil extract or fat extract of the black soldier fly (hermetia illucens); and
(e) mixing the sterilized oil extract or fat extract of the black soldier fly (hermetia illucens) with additional ingredients to form the skincare product, the additional ingredients comprising a vitamin E in a range of 1-5% by weight of the skincare product and a cosmetically-acceptable ingredient in a range of 1-100% by weight of the skincare product, wherein the pharmaceutical product contains the sterilized oil extract or fat extract of the black solder fly (hermetia illucens) is in a range from 1-30% by weight.

8. The process according to claim 7, wherein the black soldier fly (hermetia illucens) is a black soldier fly (hermetia illucens) larvae.

9. The process for preparing a skincare product according to claim 7, wherein the time is 2-5 hours.

10. The process according to claim 7, wherein the drying the black soldier fly (hermetia illucens) is done by a microwave drying for the time of 30-60 minutes.

11. The process according to claim 7, wherein the extracting the dried black solder fly (hermetia illucens) is done by the screw press.

12. The process according to claim 7, wherein the extracting the dried black solder fly (hermetia illucens) is done by the supercritical CO₂ extraction at a pressure of 200-250 bar, and at 40-60° C. for 2-3 hours.

13. The process according to claim 7, wherein the separating the oil extract or the fat extract of the black soldier fly (hermetia illucens) from the extracted mixture is done by filtration with a filter having a size of 20-30 μm.

14. The process according to claim 7, wherein the separating the oil extract or the fat extract of the black soldier fly (hermetia illucens) from the extracted mixture is done by centrifugation at 2500-4000 rpm, at 25-40° C. for 10-30 minutes.

15. The process according to claim 7, wherein the sterilizing temperature is from 60-90° C.

16. The process according to claim 7, further comprising a freezing of the black soldier fly (hermetia illucens) at a freezing temperature from −5 to −20° C. before the drying the black solder fly (hermetia illucens).

17. The process according to claim 7, further comprising a step a grinding or a milling of the dried black soldier fly obtained from step to produce a black soldier fly powder.

18. The process according to claim 17, wherein the black soldier fly powder has a size less than 1 mm.
